(19) United States
(12) Patent Application Publication
(10) Pub. No.: US 2018/0015134 A1
(43) Pub. Date: Jan. 18, 2018

(54) COMPOSITION FOR INCREASING EXERCISE PERFORMANCE ABILITY AND ENHANCING PHYSICAL STRENGTH, CONTAINING COMPOSITE EXTRACT

(71) Applicant: POSTECH ACADEMY-INDUSTRY FOUNDATION, Pohang-si, Gyeongsangbuk-do (KR)

(72) Inventors: Kyoungtai KIM, Pohang (KR); Hoeyune JUNG, Gyeongju (KR); Kwang Ho KWON, Daegu (KR)

(21) Appl. No.: 15/529,127

(22) PCT Filed: May 22, 2015
(86) PCT No.: PCT/KR2015/005169

§ 371 Date: Jul. 27, 2017

(30) Foreign Application Priority Data
Nov. 24, 2014 (KR) 10-2014-0164707

(51) Int. Cl. A61K 36/8962; A61K 36/61; A61K 36/81; A61K 36/82

(52) U.S. Cl. CPC A61K 36/8962; A61K 36/82; A61K 36/61; A61K 36/81

(57) ABSTRACT

The present invention relates to a pharmaceutical composition and a nutraceutical composition containing a composite extract of Allium sativum, Capsicum annuum, black tea and Psidium guajava as an active ingredient. According to the present invention, the pharmaceutical composition and the nutraceutical composition exhibit an excellent effect of increasing exercise performance ability and enhancing physical strength. Therefore, the composition of the present invention can be very useful in the pharmaceutical field or the nutraceutical field.

### TECHNICAL FIELD

The present invention relates to a pharmaceutical composition and a functional food composition for increasing exercise performance ability or enhancing physical strength, which contain a composite extract of Allium sativum, Capsicum annuum, black tea and Psidium guajava as an active ingredient.

### BACKGROUND ART

In complicated modern society, modern people who are exposed to the deterioration of the living environment caused by environmental pollution, an increase in mental stress, the lack of activity and the like are increasingly interested in health promotion. In order to prevent adult diseases or aging, exercise is the most effective and economical, but modern people who cannot take care of their health due to busy daily lives, fatigue and the like are interested in taking various functional foods instead of exercise. In addition, athletes use exercise-performance-enhancing supplements (ergogenic aids) to increase the efficiency of exercise performance ability in addition to scientific training and diet therapy according to the type of sport. The exercise-performance-enhancing supplement improves exercise performance ability and also is effective in removing fatigue factors which are accumulated in human bodies upon body activities and thus cause fatigue. For this reason, ordinary people as well as athletes have commonly used them.

Research on a functional supplement for improving exercise performance ability has been actively conducted in the East and West. Supplements including compounds such as a steroid, caffeine, sodium bicarbonate, sodium citrate or the like increase exercise performance ability and also provide energy in daily life. However, this is only a temporary phenomenon and may involve side effects fatal to health.

Accordingly, in recent years, the necessity to develop a functional supplement using natural products which ensure safety such as plant extracts is increasing.

Meanwhile, Allium sativum is a root vegetable belonging to the family Liliaceae and is a crop grown all over the world including Korea. Allium sativum favors a cool climate and thus it is common to sow in the fall and harvest in the spring. An Allium sativum extract is known to be used as a reactive oxygen inhibitor containing thiosulfinates as an active ingredient (Korean Patent Application Publication No. 10-2006-0039042), and be effective in preventing hyperlipidemia and abdominal obesity (Korean Patent Application Publication No. 10-2006-0067988) and suppressing the migration of tumor cells (Korean Patent Application Publication No. 10-2011-0057012).

Capsicum annuum belongs to the family Tubiflorae and is native to Central America. A Capsicum annuum extract is known to be effective in preventing and treating inflammation, allergies or asthma (Korean Patent Application Publication No. 10-2011-0106242).

Black tea is made from the leaves of Camellia Sinensis. It is known to have a strong antioxidant effect due to catechin and reduce the risk of heart disease, atherosclerosis, stroke and cancer. Also, it has been reported to be effective in treating obesity, diabetes, hyperlipidemia and fatty liver by suppressing digestion and absorption of carbohydrates and fats, reducing blood glucose, neutral fats, abdominal fat and the accumulation of neutral fats in peripheral tissue (Korean Patent Application Publication No. 10-2004-0016621).

Psidium guajava is a plant belonging to the family Myrtaceae. It has vitamins, minerals, flavonoids, tannins and the like, and thus all of leaves, bark and fruit have been used for medicinal purposes for a long time. It is native to tropical regions of the Americas and is generally distributed in subtropical regions. A Psidium guajava extract increases an anti-inflammatory activity (Korean Patent Application Publication No. 10-2013-0110398) and has efficacy as an anti-avian influenza viral agent (Korean Patent Application Publication No. 10-2011-0065534). Also, a Psidium guajava extract reduces the generation of sebum in the sebaceous gland (Korean Patent Application Publication No. 10-2014-0008335) and promotes the action of insulin, and thus is effective in suppressing an increase in blood glucose, which is a symptom of diabetes (Korean Patent Application Publication No. 10-2002-0094087).

### DISCLOSURE

### Technical Problem

The present invention has been made to develop a supplement which effectively increases exercise performance ability and has no side effects. The present invention is directed to providing a pharmaceutical composition and/or a functional food composition for increasing exercise performance ability or enhancing physical strength, which contains a composite extract of Allium sativum, Capsicum annuum, black tea and Psidium guajava as an active ingredient.

However, the scope of the present invention is not limited to the above-described objects, and other unmentioned objects may be clearly understood by those skilled in the art from the following descriptions.

### Technical Solution

The above-described objects of the present invention is accomplished by verifying the effects of increasing exercise performance ability and enhancing physical strength after a composite extract is administered to a mouse.

The present invention provides a pharmaceutical composition for increasing exercise performance ability or enhancing physical strength, which contains a composite extract of Allium sativum, Capsicum annuum, black tea and Psidium guajava as an active ingredient.

In an embodiment of the present invention, the composite extract may be extracted with one or more extraction solvents selected from the group consisting of water, methanol, ethanol, acetone, ether, petroleum ether, benzene, ethyl acetate, chloroform, mercury and liquid carbon dioxide.

In another embodiment of the present invention, the composite extract may be prepared by mixing each of Allium sativum, Capsicum annuum, black tea and Psidium guajava in a weight ratio of 1 to 95:1 to 95:1 to 95:1 to 95.

In addition, the present invention provides a functional food composition for increasing exercise performance ability or enhancing physical strength, which contains a composite extract of Allium sativum, Capsicum annuum, black tea and Psidium guajava as an active ingredient.

In an embodiment of the present invention, the composite extract may be extracted with one or more extraction solvents selected from the group consisting of water, ethanol, acetone, ether, petroleum ether, benzene, ethyl acetate, chloroform, mercury and liquid carbon dioxide.

In another embodiment of the present invention, the composite extract may be prepared by mixing each of Allium sativum, Capsicum annuum, black tea and Psidium guajava in a weight ratio of 1 to 95:1 to 95:1 to 95:1 to 95.

The pharmaceutical composition contains a composite extract and other carriers or excipients, and thus may be provided in a form which is obvious to those skilled in the art such as tablets, pills, granules, liquid and solutions, capsules and the like.

In addition, the functional food composition may be mixed with an acceptable additive upon the manufacture of food or contain other active ingredients in addition to a composite extract, and thus may be provided in a general form which is obvious to those skilled in the art such as beverages, cookies and the like.

### Advantageous Effects

A composition according to the present invention relates to a pharmaceutical composition and a functional food composition which are effective in increasing exercise performance ability and enhancing physical strength and contain a composite extract as an active ingredient. The compositions exhibit excellent effects of increasing exercise performance ability and enhancing physical strength, and thus are expected to be very usefully applied in the pharmaceutical field, the functional food field or the like.

### DESCRIPTION OF DRAWINGS

FIG. 1 is a graph illustrating effects of a composite extract on enhancement of endurance exercise performance ability and physical strength in a mouse.

FIG. 2 is a graph illustrating an effect of a composite extract on sensorimotor skill in a mouse.

FIG. 3 is a graph illustrating an effect of a composite extract on enhancement of grip strength in a mouse.

### MODES OF THE INVENTION

The present inventors verified that a composite extract of Allium sativum, Capsicum annuum, black tea and Psidium guajava is effective in increasing exercise performance ability and enhancing physical strength, and thus completed the present invention on the basis of the result.

In an embodiment of the present invention, a composite extract containing Allium sativum, Capsicum annuum, black tea and Psidium guajava was prepared (see Example 1), the composite extract thus prepared was administered to a mouse and then effects of the composite extract on enhancement of endurance exercise performance ability and physical strength were measured (see Example 2), and an effect of the composite extract on sensorimotor skill in a mouse was measured (see Example 3). Also, an effect of the composite extract on enhancement of grip strength was measured to measure a change in maximum muscle strength (see Example 4). As a result, it was confirmed that the composite extract containing Allium sativum, Capsicum annuum, black tea and Psidium guajava is effective in increasing exercise performance ability and enhancing physical strength.

Therefore, the present invention can provide a pharmaceutical composition for increasing exercise performance ability or enhancing physical strength, which contains a composite extract of Allium sativum, Capsicum annuum, black tea and Psidium guajava as an active ingredient, and a functional food composition for increasing exercise performance ability or enhancing physical strength, which contains a composite extract of Allium sativum, Capsicum annuum, black tea and Psidium guajava as an active ingredient.

The composite extract according to the present invention may be extracted with one or more extraction solvents selected from the group consisting of water, methanol, ethanol, acetone, ether, petroleum ether, benzene, ethyl acetate, chloroform, mercury and liquid carbon dioxide. The composite extract is extracted preferably with ethanol as an extraction solvent, and most preferably, with 80 to 100% ethanol as an extraction solvent, but the present invention is not limited thereto.

Also, the composite extract is preferably prepared by mixing each of Allium sativum, Capsicum annuum, black tea and Psidium guajava in a weight ratio of 1 to 95:1 to 95:1 to 95:1 to 95, but the present invention is not limited thereto.

The pharmaceutical composition according to the present invention may be prepared in a unit dose form by formulating it using pharmaceutically acceptable carriers and/or excipients according to a method which can be easily performed by those skilled in the art or by injecting it into a multi-dose container. In this case, dosage forms may be solutions in an oil or aqueous medium, suspensions, syrups, or emulsions; or extracts, powders, granules, tablets, or capsules, and a dispersant or a stabilizer may be further included.

When the pharmaceutical composition according to the present invention is formulated, diluents or excipients such as a general filler, extending agent, bonding agent, wetting agent, disintegrating agent, surfactant and the like are used.

A solid formulation for oral administration may include tablets, pills, powders, granules, capsules, troches and the like, and such a solid formulation is prepared by mixing one or more compositions according to the present invention with at least one excipient, for example, starches, calcium carbonate, sucrose, lactose, gelatin or the like. Also, lubricants such as magnesium stearate and talc are used in addition to simple excipients. A liquid formulation for oral administration may include suspensions, solutions, emulsions, syrups or the like. Various excipients such as wetting agents, sweetening agents, fragrances, preservatives and the like may be included in addition to water and liquid paraffin which are commonly employed simple diluents.

A formulation for parenteral administration may include sterilized aqueous solutions, non-aqueous solutions, suspensions, emulsions, lyophilized formulations, suppositories and the like. Examples of non-aqueous solutions or suspensions may include propylene glycol, polyethylene glycol, vegetable oil such as olive oil, an injectable ester such as ethyl oleate, and the like. Examples of suppository bases may include Witepsol, macrogol, Tween 61, cacao butter, laurin fat, glycerol, gelatin and the like.

The composition according to the present invention may be administered orally or parenterally (for example, intravenous, subcutaneous, intraperitoneal or topical application) depending upon the desired method, and the dose thereof, although varying depending on a patient′ body weight, age, gender, health condition, diet, time and route of administration, a secretion rate and severity of disease, may be appropriately selected by those skilled in the art. A daily dose of the composition is about 10 to 5,000 mg/kg body weight and is preferably divided and administered one to several times per day. However, a daily dose may be increased or decreased depending on an administration route, severity of disease, a patient′ gender, body weight, age and the like. Therefore, the dose does not limit the scope of the present invention in any respect.

The composition according to the present invention is administered in a pharmaceutically effective dose. The term “pharmaceutically effective dose” used herein refers to an amount sufficient to treat a disease at a reasonable benefit/risk ratio applicable to medical treatment, and the effective amount level may be determined by factors, including the disease type of a patient, severity, drug activity, sensitivity to a drug, administration time and route, emission rate, treatment period and co-administered drugs, and other factors well known in medicine.

The composition according to the present invention may be administered as an individual therapeutic agent or in combination with other therapeutic agents, the composition may be administered sequentially or concurrently with conventional therapeutic agents, and the composition may be administered once or multiple times. Considering all of the above factors, it is important to administer a dose that can achieve the maximum effect in a minimal amount without side effects, which may be easily determined by those skilled in the art.

In another aspect, the composition according to the present invention relates to a functional food composition for increasing exercise performance ability or enhancing physical strength. That is, the composition according to the present invention may be used simultaneously with or separately from a drug for treating a disease for increasing exercise performance ability or enhancing physical strength.

The functional food composition according to the present invention may be added to foods, beverages or the like.

The composition according to the present invention may be added to a nutritional functional food for the purpose of increasing exercise performance ability or enhancing physical strength. When the composite extract according to the present invention is used as a food additive, the composite extract may be added without change or used together with other foods or food ingredients, and appropriately used according to a conventional method. The amount of the active ingredients to be mixed may be appropriately determined according to their intended use (e.g., prevention, health or therapeutic treatment). Generally, upon manufacture of foods or beverages, the composite extract according to the present invention is added in an amount of 15 wt % or less, preferably, 10 wt % or less with respect to a raw material. However, in the case of long-term intake for health and hygiene or for regulating health, the amount may be below the above range, and since there is no problem in terms of safety, an active ingredient may also be used in an amount exceeding the above range.

A type of the food is not specifically limited. Examples of foods to which the composition can be added include meat, sausages, bread, chocolate, candies, snacks, cookies, pizza, ramen, other noodles, chewing gum, dairy products including ice cream, various types of soup, beverages, tea, health drinks, alcoholic drinks, vitamin complexes and the like, and include all nutritional functional foods in a conventional sense.

Components of a nutritional functional food according to the present invention are not limited, other than that the composition be contained as an essential ingredient in an indicated percentage, to other components, and may contain various flavoring agents, natural carbohydrates or the like as an additional component like conventional beverages. Examples of the natural carbohydrates include monosaccharides, for example, glucose, fructose and the like; disaccharides, for example, maltose, sucrose and the like; and polysaccharides, for example, conventional sugars such as dextrin, cyclodextrin and the like, and sugar alcohols such as xylitol, sorbitol, erythritol and the like. In addition to the above-described compounds, natural flavoring agents (thaumatin and stevia extracts (e.g., rebaudioside A, glycyrrhizin, etc.)) and artificial flavoring agents (saccharin, aspartame, etc.) may be beneficially used as a flavoring agent. The percentage of the natural carbohydrates may be appropriately determined by those skilled in the art.

In addition to the above-described compounds, the functional food composition according to the present invention may contain various nutrients, vitamins, minerals (electrolytes), flavoring agents such as artificial flavoring agents, natural flavoring agents and the like, coloring agents and improving agents (cheese, chocolate, etc.), pectic acid and salts thereof, alginic acid and salts thereof, organic acids, protective colloidal thickening agents, pH controlling agents, stabilizers, preservatives, glycerin, alcohols, carbonizing agents as used in carbonated beverages or the like. In addition, the composition according to the present invention may contain flesh for manufacturing natural fruit juice, fruit juice beverages and vegetable beverages. The components may be used alone or in combination. The percentage of such an additive may also be appropriately determined by those skilled in the art.

Hereinafter, exemplary examples will be described for promoting understanding of the present invention. However, the following examples should be considered in a descriptive sense only, and the scope of the invention is not limited to the following examples.

### EXAMPLE

The present invention is composed of Example 1 in which a composite extract is prepared, Example 2 in which effects of the composite extract on enhancement of endurance and physical strength are measured, Example 3 in which a sensorimotor skill effect is measured, and Example 4 in which maximum muscle strength is measured. Through the above examples, the new use of the composite extract according to the present invention will be evaluated. Hereinafter, the present invention will be described in detail.

### Example 1. Preparation of Composite Extract Containing Allium sativum, Capsicum annuum, Black Tea and Psidium guajava

100 g of Allium sativum, 100 g of Capsicum annuum and 100 g of Psidium guajava were washed and chopped using a mixer. The chopped Allium sativum, Capsicum annuum and Psidium guajava were extracted using 1 L of 100% ethanol (E1281, Samchun Pure Chemical) at room temperature for 48 hours, and then an extract solution was filtered with filter paper having a diameter of 300 mm (Toyo Roshi Kaisha, Ltd., Japan) to remove impurities. Afterward, the resulting substance was concentrated under a reduced pressure using a rotary vacuum evaporator (EYELA, Tokyo Rikakikai Co., Ltd., Japan), and freeze-dried. Black tea in a powder form was used. 30 g of black tea was extracted using 1 L of 80% ethanol at room temperature for 48 hours, and then an extract solution was filtered with filter paper having a diameter of 300 mm to remove impurities. Afterward, the resulting substance was concentrated under a reduced pressure using a rotary vacuum evaporator, freeze-dried, and then used in effect experiments.

### Example 2. Measurement of Effects of Composite Extract on Enhancement of Endurance Exercise Performance Ability and Physical Strength in Mouse

2-1. Administration of Composite Extract and Negative Control

7 week old ICR mice were purchased from Koatech, pre-fed for 1 week, and then divided into two groups of 10 mice each.

First, phosphate-buffered saline (PBS) was administered to a first group, which was set as a negative control, and the composite extract was orally administered to a second group through a 1 ml syringe every day for 4 weeks.

2-2. Measurement of Effects of Enhancing Endurance Exercise Performance Ability and Physical Strength in Mouse

In order to measure a change in endurance exercise performance ability in a mouse, caused by the composite extract, treadmill exercise (Panlab & Harvard, Spain) was performed for animals of exercise performance groups. A protocol for the exercise performance was devised to consisting of a combination of two sets of running on a slope of 5 degrees at a speed of 25 cm/sec for 20 minutes, at a speed of 30 cm/sec for 20 minutes, at a speed of 33 cm/sec for 20 minutes and at a speed of 36 cm/sec for 15 minutes, and running on a slope of 10 degrees at a speed of 38 cm/sec for 15 minutes and at a speed of 41 cm/sec until the animals were exhausted. On the day before the experiment, in order to adapt to the treadmill running, the animals exercised on a slope of 5 degrees at a speed of 27 cm/sec for 10 minutes. On the day of the experiment, the maximum exercise performance ability was measured after experimental animals fasted 2 hours before treadmill running A determination point of the maximum exercise performance ability was defined as a point at which the experimental animals could not catch up with a treadmill speed for 20 seconds or more after the start of exercise or the accumulated number of electric shocks exceeded 100 times within 5 minutes. The experimental results verified significance through a t-test for the experimental group and control, and showed a statistically significant difference (**p<0.005, ***p<0.0005).

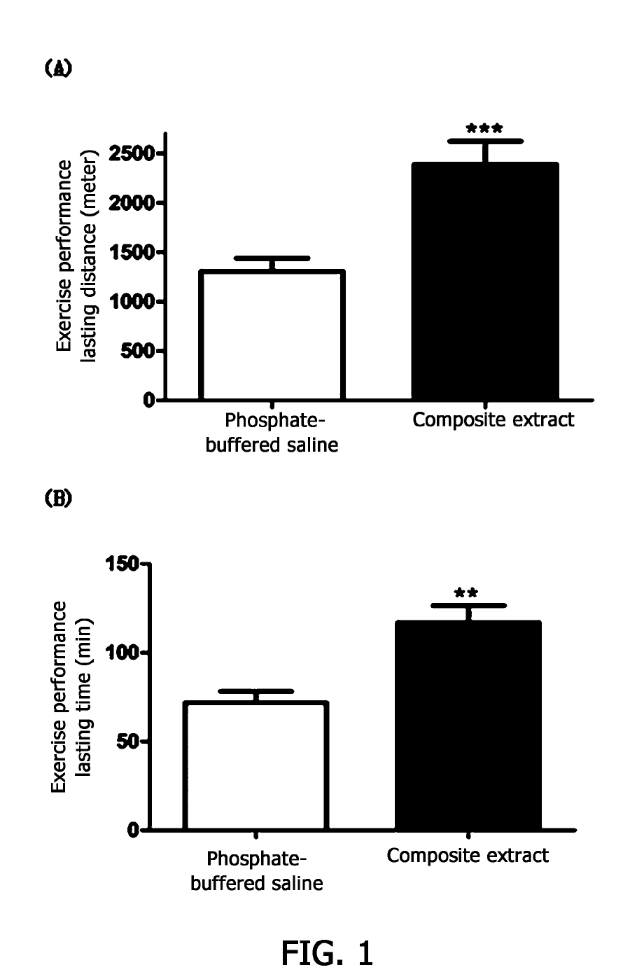

As a result, as shown in FIG. 1, an exercise performance lasting time of a composite-extract-administered group was significantly increased compared to a PBS-administered group. Therefore, it was confirmed that the intake of a composite extract highly contributed to enhancing physical strength, and the composite extract according to the present invention can be used as a supplement for enhancing physical strength.

### Example 3: Measurement of Effect of Composite Extract on Sensorimotor Skill in Mouse

3-1. Administration of Composite Extract and Negative Control

7 week old ICR mice were purchased from Koatech, pre-fed for 1 week, and then divided into two groups of 10 mice each. As in Example 2-1, a first group was set as a negative control (administration of PBS), and the composite extract was orally administered to a second group through a 1 ml syringe every day for 4 weeks.

3-2. Measurement of Sensorimotor Skill Effect in Mouse

In order to measure an effect of the composite extract on sensorimotor skill in a mouse, a rotarod test (Panlab & Harvard, Spain) for acceleration was performed for the animals of the control and experimental groups. The rotarod test for acceleration is intended to measure the motor coordination and balance of animals.

After holding the tail of a mouse and placing it on a rotarod, a speed was increased by 4 rpm every 30 seconds from 4 rpm to 40 rpm within 5 minutes, and a time in which a mouse stayed on a rotarod was measured. This process was repeated five times and the longest time that a mouse stayed on a rotarod was used as a result. The experiment results verified significance through a t-test for the experimental group and control and showed a statistically significant difference (*p<0.05).

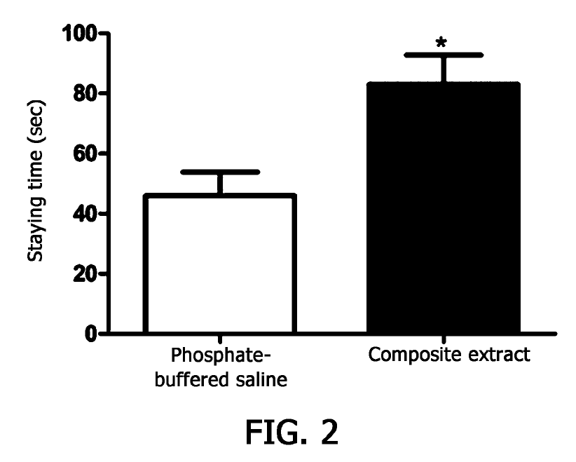

As a result, as shown in FIG. 2, the time in which the mouse stayed on a rotarod was significantly longer in the composite-extract-administered group compared to the PBS-administered group. Therefore, it can be seen that the intake of the composite extract significantly increases sensorimotor skills, and the composite extract according to the present invention was effective in enhancing motor coordination and balance.

### Example 4: Measurement of Effect of Composite Extract on Enhancement of Grip Strength in Mouse

4-1. Administration of Composite Extract and Negative Control

20 7-week-old ICR mice having a body weight of 28±0.5 g were divided into two groups of 10 mice each. As in Example 2-1 and Example 3-1, a first group was set as a negative control (administration of PBS), and the composite extract was orally administered to a second group through a 1 ml syringe every day for 4 weeks.

4-2. Measurement of Effect of Enhancing Grip Strength in Mouse

In order to measure a change of maximum muscle strength caused by a composite extract, a grip strength test (Bioseb, France) was performed for the animals of the control and experimental groups. After holding the tail of a mouse and allowing the mouse to grab an apparatus for testing grip strength, the maximum grip strength of front and hind feet when the tail was pulled backwards was measured. Grip strength was repeatedly measured three times, and then an average value thereof was used. The experiment results verified significance through a t-test for the experimental group and control and showed a statistically significant difference (*p<0.05).

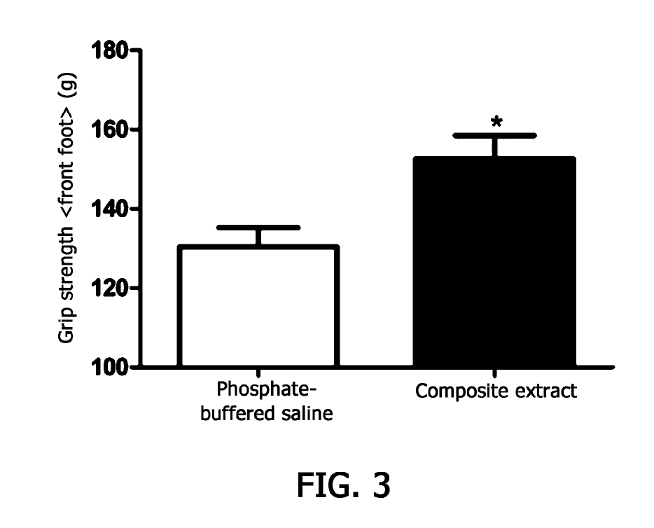

As a result, as shown in FIG. 3, the maximum muscular strength (grip strength) of the composite-extract-administered group was increased compared to the PBS-administered group. Therefore, it can be seen that the intake of the composite extract significantly contributed to enhancing muscular strength, and the composite extract according to the present invention was effective in enhancing muscular strength.

The above description of the present invention is only exemplary, and it will be understood by those skilled in the art that various modifications can be made without departing from the scope of the present invention and without changing essential features. Therefore, the above-described embodiments should be considered in a descriptive sense only and not for purposes of limitation.

1. A method of increasing exercise performance ability or enhancing physical strength, the method comprising:
administering, to a subject in need thereof, a composite extract of Allium sativum, Capsicum annuum, black tea and Psidium guajava as an active ingredient.

2. The method of claim 1, wherein the composite extract is extracted with an extraction solvent selected from the group consisting of water, methanol, ethanol, acetone, ether, petroleum ether, benzene, ethyl acetate, chloroform, mercury and liquid carbon dioxide.

3. The method of claim 1, wherein the composite extract is prepared by mixing each of Allium sativum, Capsicum annuum, black tea and Psidium guajava in a weight ratio of 1 to 95:1 to 95:1 to 95:1 to 95.

4. (canceled)

5. (canceled)

6. (canceled)
